(19) United States
(12) Patent Application Publication
(10) Pub. No.: US 2026/0053419 A1
(43) Pub. Date: Feb. 26, 2026

(54) SYSTEMS AND METHODS FOR DETECTION OF STROKE

(71) Applicant: REMORPHOSIS SOLUTIONS PRIVATE LIMITED, Mumbai (IN)

(72) Inventors: Rachit Shailain Jhaveri, Mumbai (IN); Alok Raj Aggarwal, Mumbai (IN)

(21) Appl. No.: 19/378,314

(22) Filed: Nov. 3, 2025

Related U.S. Application Data
(63) Continuation of application No. PCT/IN2024/050463, filed on May 1, 2024.

(30) Foreign Application Priority Data
May 1, 2023 (IN) 202321031128

(51) Int. Cl. A61B 5/369; A61B 5/00; A61B 5/265; A61B 5/305; A61B 5/374

(52) U.S. Cl. CPC A61B 5/369; A61B 5/265; A61B 5/305; A61B 5/374; A61B 5/7257

(57) ABSTRACT

Systems and methods for detection of stroke and its types, comprising: electrodes, on an EEG headset (101), placed to record EEG signals; a client-side signal processing engine configured to: compute power for each of signals; segregate processed signal, from each of said electrodes, into five baskets, by processing signals from each of said electrodes such that there is a Delta basket, a Theta basket, an Alpha basket, a Beta basket extract features from a frequency component of said transformed signals in order to obtain stroke ratios; receive, as a first output, a first set of processed signals with power ratings for determination of a stroke incident as a function of power rating ratios; receive, as a second output, a second set of processed signals with relative powers for determination of a type of stroke as a function of said first relative power (RDP) and said second relative power (RAP).

### RELATED APPLICATIONS

This application claims priority under 35 U.S.C. § 120 to, and is a continuation of, co-pending International Application PCT/IN2024/050463, filed May 1, 2024 and designating the US, which claims priority to IN Application No. 202321031128, filed May 1, 2023, such IN application also being claimed priority to under 35 U.S.C. § 119. These IN and International applications are incorporated by reference herein in their entireties.

### FIELD

This invention relates to the field of biomedical engineering. Particularly, this invention relates to systems and methods for detection of stroke.

### BACKGROUND

Stroke is the fifth leading cause of death and the leading cause of preventable disability in the United States of America as per some reports. Out of all low- and middle-income countries, in India, stroke is one of the leading causes of disability and death.

From the onset of a stroke, a patient has a short window period (about 8-12 hours from onset of symptoms) during which interventions can be performed (surgically or using drugs) in order to minimize damage caused by loss of blood supply to the brain because of the clot.

Within a window period of 3-4.5 hours, there are possibilities of irreversible damage to the brain and chances of favourable outcomes by thrombolysis with administration of intravenous alteplase is about 50% (Hacke W, 2008; Thrombolysis with alteplase 3 to 4.5 hours after acute ischemic stroke. N Engl J Med.).

Globally, majority of patients who suffer a stroke visit a general physician (Nguyen, 2021; Stroke patient's alarm choice: General practitioner or emergency medical services. Acta Neurol Scand. 2021; 143:164-170) (F Johnston, 1999; Delays in stroke referral. Lancet) (Joseph Kwan, P. H. (2004; A systematic review of barriers to delivery of thrombolysis for acute stroke. Age Ageing) and the symptoms being similar to conditions other than stroke, the patient is made to wait which delays the process of administering tPA (F Johnston, 1999).

Faster diagnosis of stroke, in the field, would allow paramedics to alert hospitals sooner and have stroke specialists immediately available when a patient arrives. A similar approach has been used, for years, in cases of trauma, which allows specialized teams to be ready when a severely injured patient arrives.

Currently, a patient who exhibits symptoms of stroke is clinically assessed and is sent for a CT scan. However, a CT scan may not show an infarct up to 6-8 hours of onset of the stroke.

MRI is a valuable imaging tool during a stroke, however the numbers of nursing homes, diagnostic centres, hospitals, and/or the like, providing an MRI scanner are few in metropolitan cities and this number steeply drops in rural areas. Another drawback of an MRI scan is the time it takes for a procedure to be complete, including the transit of patient from an emergency room to a radiology department that houses an MRI scanner.

If the CT scans do not definitely display an infarct, putting the patient on thrombolysis is not possible since performing intravenous thrombolysis on hemorrhagic stroke patients is considered an absolute contraindication since risks of bleed and death overweigh possible benefits of administering it (Jennifer E. Fugate, D. a. 2015; Absolute and Relative Contraindications to IV rt-PA for Acute Ischemic Stroke. The Neurohospitalist).

And, thus, a large patient population is either withheld from any administration of Iv-tPA until brain imaging results confirm presence of an ischemia, ruling out a bleed or are administered Iv-tpA with an assumption of an Ischemia after ruling out hemorrhage, without ruling out stroke mimics (O. Y. Chernyshev, 2010; Safety of tPA in stroke mimics and neuroimaging-negative cerebral ischemia. Neurology). It may also take a while for a patient to reach a multi-specialty hospital to get diagnosed with stroke due to common trends of going to a general practitioner for initial assessment of the condition.

### SUMMARY

Thus, a sensitive and specific device is required which can detect if the patient has stroke, differentiating it from a stroke mimic, and type of the stroke. Also, ambulance, hospital, and neurosurgeons need to be informed about the case, in case of detection, so as to be ready for further tests and treatment of the patient.

Prior art EEG based devices and processes are made to measure neural electrical signals which can be broadly classified into two categories:
conventional systems—EEG systems available in clinical settings; and
brain-sensing headsets: Neurosky, Muse, Emotiv, etc.

Electroencephalographic (EEG) signals serve as an important source of information when it comes to brain functionality. Abnormal activities of the brain can be recognized using EEG rhythms. Most of the cerebral signal observed in the scalp, using surface EEG (sEEG) falls in the range of 1-20 Hz. Waveforms are subdivided into bandwidths known as delta (δ), theta (θ), alpha (α), and beta (β) to signify the majority of the EEG used in clinical practice (Claassen, 2012; Quantitative EEG for the detection of brain ischemia. Critical care 16, 2 (2012), 216). Ischemic stroke is primarily because of changes in Cerebral Blood Flow (CBF), and it can be detected through changes in EEG signal patterns (Jan W Kantelhardt, S. A.-B. 2002; Multifractal detrended fluctuation analysis of nonstationary time series. Physica A: Statistical Mechanics and its Applications 316, 1-4 (2002), 87-114). Prominent changes include increase of delta (lowest frequency band) and/or attenuation of high-frequency bands (beta and alpha) (Claassen, 2012), (Simon Finnigan, A. W. 2016; Defining abnormal slow EEG activity in acute ischaemic stroke: Delta/alpha ratio as an optimal QEEG index. Clinical Neurophysiology 127, 2 (2016), 1452-1459).

Furthermore, the power density ratio between bands of the different hemisphere changes as stroke affects one hemisphere (Claassen, 2012), (Simon Finnigan, 2016), (Zhiyong Liu, J. S. 2016; Sleep staging from the EEG signal using multi-domain feature extraction. Biomedical Signal Processing and Control 30 (2016), 86-97). This data can be computed using the pairwise-derived brain symmetry index (pdBSI) which is used to identify the differences in EEG-quantified interhemispheric cortical power asymmetry observable in stroke patients. Since these parameters are correlated with the biochemical changes associated with stroke (Gratianne Rabiller, 2015; Perturbation of Brain Oscillations after Ischemic Stroke: A Potential Biomarker for Post-Stroke Function and Therapy. Int. J. Mol. Sci), changes observed in these parameters are almost instant from the onset of symptoms, sometimes even before the changes in the brain can be visualized using imaging techniques (Finnigin et al., 2004, 2006, 2008).

Thus, the present invention aims at providing a screening tool for early detection of stroke, differentiating it from stroke-mimics, and distinguishing between stroke subtypes namely Ischemic and Hemorrhagic stroke.

Stroke occurs when the blood supply to the brain is obstructed, either due to a blockage, referred to as an Ischemic attack or due to a bleed, referred to as a hemorrhagic attack. Symptoms linked with a stroke typically constitute of hemiparesis, loss of speech, headache and movement related disorders. However, one of the immediate symptoms are headache and slight disorientation. The symptoms lacking specificity to stroke are more often misdiagnosed as stroke mimics. Quite often, stroke mimics are also diagnosed as stroke. It therefore becomes imperative that the correct diagnosis is made. Specialists and trained physicians in stroke can clinically identify a stroke, but to distinguish a hemorrhagic stroke from an ischemic stroke requires the aid of an imaging tool like Computed Tomography (CT) or a Magnetic Resonance Imaging technique. The availability, cost, and time for testing associated with these gold standard imaging tools poses a need for a point of care device that can detect early signs of a stroke and aid as a screening tool for stroke.

Electroencephalography (EEG) has been a gold standard for capturing volumetric brain signals for epileptic patients. The use of EEG in stroke analysis has been studied for over two decades. The underlying principle being that the loss of blood supply to the brain causes changes in the electrical activity within the brain, which can be captured using electroencephalography.

A comparison of the existing brain-sensing headsets and the current invention can be tabulated as under:

| | | | | Use case | | | | |
|---|---|---|---|---|---|---|---|---|
| | | Sampling | | and | | | | |
| Device | Electrodes | Rate | | availability | End use | Cost | | Reference |
| InteraXon | Rigid electrode | 256 | Hz | Research | Focus and | NA | | Doudou et |
| | placement | | | tools for | Meditation | | | al., 2018 |
| | | | | Windows, Mac, | | | | |
| | | | | and Linux | | | | |
| Muse v1, | 4 channels | 12 | bits | Source Developer | Focus and | $200 | USD | |
| v2 | AF7, AF8, | | | Kit for Android, | Meditation | | | |
| | TP9, TP10 | | | IOS, Windows | | | | |

| | | | | | | | |
|---|---|---|---|---|---|---|---|
| Neurosky | Rigid electrode | 512 Hz | SDK | EEG power spectrum | $99.99 | USD | Doudou et |
| MindWave | placement | 12 bits | available | | | | al., 2018 |
| | 1 Channel AFz | | | | | | |

| | | | | | | |
|---|---|---|---|---|---|---|
| Open BCI | Upto 16 Channels | 256 Hz | Open-Source | Biosensing and | $500 USD for 8 | Doudou et |
| | Flexible electrode | 24 bits | Software, | Neurofeedback | channels, | al., 2018 |
| | placement at 35 | | Firmware, | | $949 USD for 16 | |
| | locations | | and hardware | | | |
| Emotiv | Rigid electrode | 128 Hz | Research | Thoughts, Feelings | $799 USD (Epoc), | Doudou et |
| Epoc, Flex, | placement | 14 bits | tools for | and expressions | $299 USD (Insight) | al., 2018 |
| and | Epoc: 14 channels | | Windows, Mac, | | | |
| Insight | AF3, F7, F3, FC5, | | and Linux | | | |
| | T7, P7, O1, O2, | | | | | |
| | P8, T8, FC6, F4, | | | | | |
| | F8, AF4 | | | | | |
| | Insight: 5 channels | | | | | |
| | AF3, AF4, T7, T8, Pz | | | | | |
| Remorphosis | Rigid and flexible | 256 Hz | Clinical | Computes Stroke or | | |
| | electrode placement | 12 bits | and | Stroke mimic and | | |
| | 4 rigid channels | | Research | Stroke-type (Ischemic | | |
| | T3, T4, O1, O2 | | | or hemorrhagic) and | | |
| | | | | continuous | | |
| | | | | long-term monitoring | | |

According to prior art, standard high-end EEG systems are provided by Mitsar in their Smart BCI EEG headset. This headset contains 16-64 electrodes. The advantage of the Smart BCI EEG headset is that it provides large number of channels (16-64 channels), Higher ADC resolution (16-24 bits), Rechargeable power source, on-board storage, dedicated software. The disadvantage being the system is wired, with high set-up time. The cost of the product is high and is not affordable in rural and emergency room set-up.

There is a need to bridge the gap between low-end wireless EEG systems and high-end EEG systems by providing a solution which incorporates high accuracy in terms of ADC resolution, point of care “standalone” system.

Prior art is plagued with a variety of problems, listed below:
Absence of screening tool to detect stroke: According to prior art, there is no screening tool for detection of stroke available for general practitioners, ambulatory services, and/or clinicians in an emergency room. The clinicians, currently, inspect for clinical symptoms of stroke and send suspected patients for a CT/MRI. The load on CT/MRI increases in case of detecting a stroke-mimic instead of a stroke. Also, the availability of CT/MRI machines is limited in urban as well as rural locations.
Absence of a stand-alone point-of-care system for rapid diagnosis of stroke and distinguishing between Ischemic and Hemorrhagic Stroke: The current gold standard for identifying a stroke is MRI. Due to drawbacks of MRI (Availability, large testing time, large patient queue, cost) (Qureshi, A. A. 2018; Ischemic Stroke Detection using EEG Signals. CASCON'18), patients/clinicians are unable to either accurately identify a stroke or provide the necessary treatment due to unavailability of the diagnostic tool. Thus, there is a requirement of a stand-alone, compact, screening tool, for accurate early detection of stroke and its subtypes to provide the required treatment.
Non-administration of tPA: Clinicians evaluating patients who clinically show strong symptoms of stroke are faced with another challenge of identifying stroke subtype. The line of treatment for Ischemic and Hemorrhagic strokes is polar opposite. Ischemic strokes would require the administration of tPA within 3-4.5 hours of onset of symptoms and a further thrombectomy procedure within 9 hours of onset of symptoms. Hemorrhagic stroke, on the other hand, would require a completely different procedure of facilitating clotting of blood. In order to take correct decisions, clinicians enforce validation of an imaging technique like CT/MRI, which consumes time and is not freely available. Thus, there is a need of a device that can accurately distinguish between an Ischemic stroke and a hemorrhagic stroke in order to facilitate the administration of tPA to ischemic stroke patients.

An object of the invention is to provide systems and methods configured for detection of stroke versus stroke mimic.

Yet an additional object of the invention is to provide a stand-alone, compact, screening tool, for accurate early detection of stroke and its subtypes to provide the required treatment.

Still an additional object of the invention is to provide a device that can accurately distinguish between an Ischemic stroke and a hemorrhagic stroke in order to facilitate the administration of tPA to ischemic stroke patients.

Another object of the invention is to design and develop a non-invasive point of care device for stroke monitoring (for immediate and long-term use cases along with any other stroke-related use cases), the device to be based on electroencephalography (EEG) whereby the aforesaid device captures neural electrical signals using 8 electrodes (including the reference and the ground electrode).

Yet another object of the invention is to design and develop a non-invasive point of care device for stroke monitoring, the device being of relatively higher accuracy for clinical use.

Still another object of the invention is to bridge the gap between low-end wireless EEG systems and high-end EEG systems by providing a solution which incorporates high accuracy in terms of ADC resolution, point of care “standalone” system.

An additional object of the invention is to reduce setup time and test time for detection of stroke.

According to this invention, there are provided A systems and methods for detection of stroke, in order to differentiate between stroke and stroke mimics, said system being an electroencephalography (EEG) signal-based system, said system comprising:
electrodes, on an EEG headset, placed to record EEG signals, for pre-defined time durations, according to known systems of electrode placement, each electrode having at least a permanent part segment and a disposable electrode segment;
a client-side signal processing engine configured to derive frequency dependent ratios from said recorded EEG signals, said client-side signal processing engine configured with instructions in order to:
   receive signals from each of said electrodes;
   amplify said recorded EEG signals to obtain amplified signals;
   divide said amplified signals into epochs on which Fast Fourier Transform is performed in order to obtain transformed signals;
   process said received signals, through a signal conditioning circuit, comprising identical signal condition blocks having a high rejection ratio (CMRR), in order to output:
      amplified signals;
      signals with attenuated high frequency noise parameters, these signals being processed signals;
   receive independent analog-to-digital (ADC) channels for receiving corresponding signals from said signal conditioning circuit, in order to:
      compute power for each of said processed signals;
      segregate processed signal, from each of said electrodes, into five baskets, by processing signals from each of said electrodes such that
         a Delta basket adds all the power values from 0 Hz to 4 Hz with their power values obtained at intervals of 0.25 Hz—with a relative power spectral band, for the Delta basket, being in the region of 0-4 Hz,
         a Theta basket adds all the power values from 4 Hz to 8 Hz with their power values obtained at intervals of 0.25 Hz—with a relative power spectral band, for the Theta basket, being in the region of 4-8 Hz,
         an Alpha basket adds all the power values from 8 Hz to 12 Hz with their power values obtained at intervals of 0.25 Hz—with a relative power spectral band, for the Alpha basket, being in the regio of 8-12 Hz,
         a Beta basket adds all the power values from 12 Hz to 30 Hz with their power values obtained at intervals of 0.25 Hz—with a relative power spectral band, for the Beta basket, being in the regio of 12-30 Hz,
   extract features from a frequency component of said transformed signals in order to obtain stroke ratios;
   receive, as a first output, a first set of processed signals with power ratings being:
      a first power rating ratio (DTABR) as a function of ratios of said delta basket and said theta basket to said alpha basket and said beta basket;
      a second power rating ratio (DAR) as a function of ratios of said delta basket and said alpha basket;
      determination of a stroke incident as a function of said first power rating ratio and said second power rating ratio;
   receive, as a second output, a second set of processed signals being:
      a first relative power (RDP) as a function of powers of signals from said delta basket;
      a second relative power (RAP) as a function of powers of signals from said alpha basket;
      determination of a type of stroke as a function of said first relative power (RDP) and said second relative power (RAP); and
a dashboard configured to receive data from said client-side signal processing engine at said headset by means of a communication protocol.

In at least an embodiment, said EEG headset comprising at least six signal electrodes, one reference electrode, and one bias electrode.

In at least an embodiment, said EEG headset comprising four fixed signal electrodes, two variable signal electrodes one reference electrode, and one bias electrode.

In at least an embodiment, said disposable electrode segment being an Ag/AgCl electrode snapped in a permanent socket made of highly conductive material to ensure lossless transmission of the EEG signal.

In at least an embodiment, said amplification being in the region of 10,000 times to maintain fidelity of signals.

In at least an embodiment, said client-side signal processing engine comprising a pre-processing engine configured with instructions in order to perform the steps of:
first-stage pre-processing, comprising a high precision amplifier with its gain set by setting external resistor values between gain ranges from 1 to 10,000;
second-stage pre-processing, and third-stage pre-processing comprising amplification stages, filters removing unwanted high frequency noise, said filters being designed to operate between 0.16 Hz and 99.98 Hz; and
filtering, for use, signals with regions of interest of frequencies being in the range from 0.1 Hz to 100 Hz.

In at least an embodiment, said client-side signal processing engine comprising a pre-processing engine configured with instructions in order to perform the steps of:
first-stage pre-processing, comprising a high precision amplifier with its gain set by setting external resistor values between gain ranges from 1 to 10,000, said amplifier comprising:
   Active High pass filter circuit contributing to the amplification of the signal from input signal in the range of 100 uV to output signal in the range of 1 mV;
second-stage pre-processing, and third-stage pre-processing comprising amplification stages, filters removing unwanted high frequency noise, said filters being designed to operate between 0.16 Hz and 99.98 Hz, said filters comprising:
   combination of bandpass filter, notch filter and non-inverting amplifier, said bandpass filter being realised by a passive high pass filter 303 having a cut-off frequency again of 0.1591 Hz and a Low pass filter 305 having a cut-off frequency of fc=96.45 Hz, said notch filter and said non-inverting amplifier 304 being sandwiched in between a high pass filter 303 and a low pass filter 305, said notch filter configured to remove electrical line noise of 50 Hz and said non-inverting amplifier configured to amplify said signals with a gain of 18.434, said combination of filters (bandpass filter, notch filter, and non-inverting amplifier) providing a gain of 12.10.

In at least an embodiment, said processed signals, from each of said six electrodes being classified into following spectral bands:
a delta spectral band, which adds all power values from 0 Hz to 4 Hz (power values obtained at intervals of 0.25 Hz),
a theta spectral band, which adds all power values from 4 Hz to 8 Hz,
an alpha spectral band which adds all power values from 8 Hz to 12 Hz,
a beta spectral band which adds all power values from 12 Hz to 30 Hz, and
a total power spectral band, which adds all power values from 0 Hz to 30 Hz.
In at least an embodiment, server-side signal processing engine comprising a processor with instructions to perform the steps of:
determining relative power for each of spectral bands (Delta: 0-4 Hz; Theta: 4-8 Hz; Alpha: 8-12 Hz; Beta: 12-30 Hz) for each channel, each channel having six electrodes;
obtaining relative powers by normalizing with a total power across the 0-30 Hz range;
computing a first ratio being a DTABR ratio;
computing a second ratio being a DAR ratio; and
averaging relative power for each spectral bank, each of said first ratios and each of said second ratios;
computing a stroke index (SI) as a weighted function of said first ratio and said second ratio.

In at least an embodiment, said client-side signal processing engine comprising a pre-processing engine configured with instructions in order to perform the steps of:
first-stage pre-processing, comprising a high precision amplifier with its gain set by setting external resistor values between gain ranges from 1 to 10,000, said amplifier comprising:
   Active High pass filter circuit 302 contributing to the amplification of the signal from input signal in the range of 100 uV to output signal in the range of 1 mV;
second-stage pre-processing, and third-stage pre-processing comprising amplification stages, filters removing unwanted high frequency noise, said filters being designed to operate between 0.16 Hz and 99.98 Hz, said filters comprising:
   combination of bandpass filter, notch filter and non-inverting amplifier, output of the High pass active filter 403 is sent first to another high pass filter with a cut-off frequency of 0.1591 Hz, followed by a notch filter of 50 Hz and a non-inverting amplifier, followed by a low pass filter with a cut-off frequency of 96.45 Hz.

In at least an embodiment, said client-side signal processing engine comprising a pre-processing engine configured with instructions in order to perform the steps of:
first-stage pre-processing, comprising a high precision amplifier with its gain set by setting external resistor values between gain ranges from 1 to 10,000, said amplifier comprising:
   Active High pass filter circuit 302 contributing to the amplification of the signal from input signal in the range of 100 uV to output signal in the range of 1 mV;
second-stage pre-processing, and third-stage pre-processing comprising amplification stages, filters removing unwanted high frequency noise, said filters being designed to operate between 0.16 Hz and 99.98 Hz, said filters comprising:
   combination of bandpass filter, notch filter and non-inverting amplifier, the AC gain of the high pass filter is 0.9968, the AC gain of the notch filter and non-inverting amplifier is 18.434 and the AC gain of the low pass filter is 0.6585.

### BRIEF DESCRIPTION OF THE ACCOMPANYING DRAWINGS

The invention will now be described in relation to the accompanying drawings, in which:

FIG. 1 contains the System Level Block Diagram. This figure depicts the major components available to the user;

FIG. 2 depicts the Board Level Block Diagram. The heart of the headset is the circuit. This figure provides a detailed explanation of the sections of the circuit;

FIG. 3 depicts the block diagram of a single signal conditioning circuit;

FIG. 4 exhibits the signal conditioning circuit;

FIG. 5 displays the output of the Instrumentation Amplifier with an input of the raw EEG signal;

FIG. 6 displays the output of the active high pass filter;

FIG. 7 displays the output of the signal condition block—the signal from here is transmitted to the microcontroller for further processing and analysis; and

FIG. 8 shows a step-by-step flow chart of the algorithm to display the conclusive parameters.

FIG. 9 shows the rachet mechanism deployed in the headset to accommodate the movable electrodes.

### DETAILED DESCRIPTION

According to this invention, there are provided systems and methods for detection of stroke.

Differentiating Stroke from Stroke mimics may be difficult (Vellieux, 2021; Spectral analysis of EEG in etiological assessment of patients with transient neurological deficits. Elsevier Masson). Differential diagnosis primarily depends on brain imaging techniques (Krishnaswamy A, 2010; Clinical Cerebrovascular anantomy. Catheter Cardiovasc Interv). Brain Magnetic Resonance Imaging (MRI) is a gold standard for Transient Neurological Disorders workup but it is usually normal, especially in the case of Transient Ischemic attack (Souillard-Scemama R, Tisserand M, Calvet D, Jumadi, 2015; 42:3-11; An update on brain imaging in transient ischemic attack. J Neuroradiol.) The primary feature of the present invention is the ability of the device to provide rapid analysis to differentiate between stroke and stroke mimics.

The device is based on electroencephalography (EEG) whereby the aforesaid device captures neural electrical signals using 8 electrodes (including the reference and the ground electrode). These electrodes are placed according to the 10-20 international system of electrode placement for EEG.

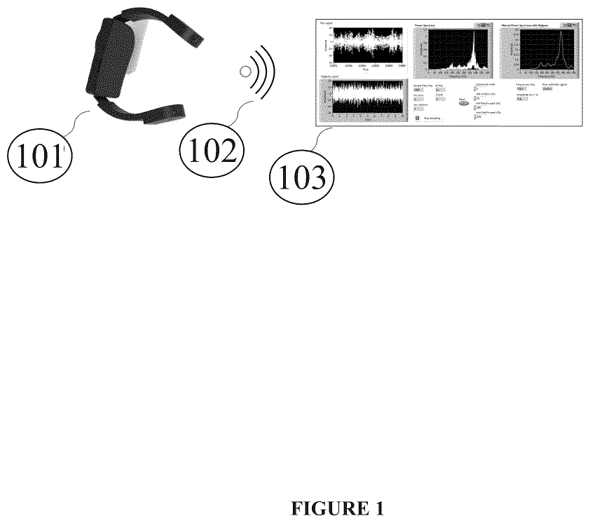

FIG. 1 contains the System Level Block Diagram. This figure depicts the major components available to the user.

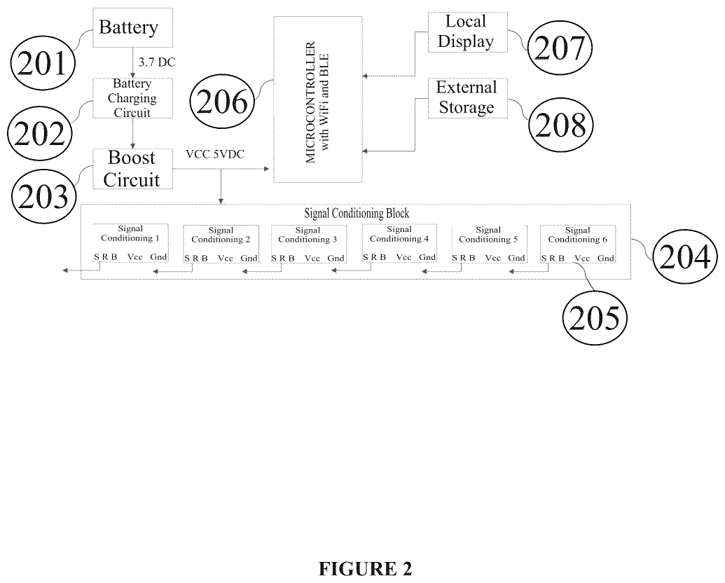

FIG. 2 depicts the Board Level Block Diagram. The heart of the headset is the circuit. This figure provides a detailed explanation of the sections of the circuit.

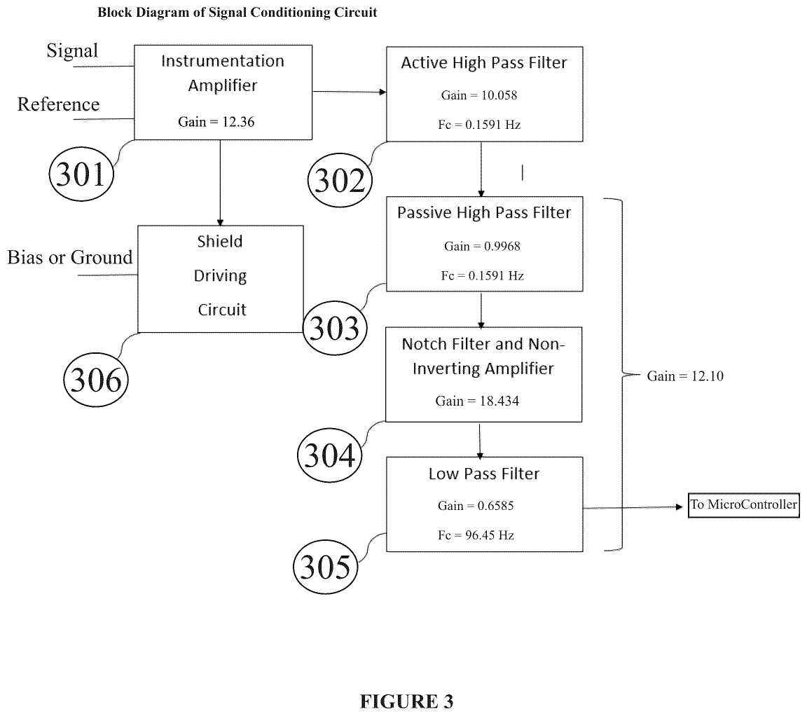

FIG. 3 depicts the block diagram of a single signal conditioning circuit.

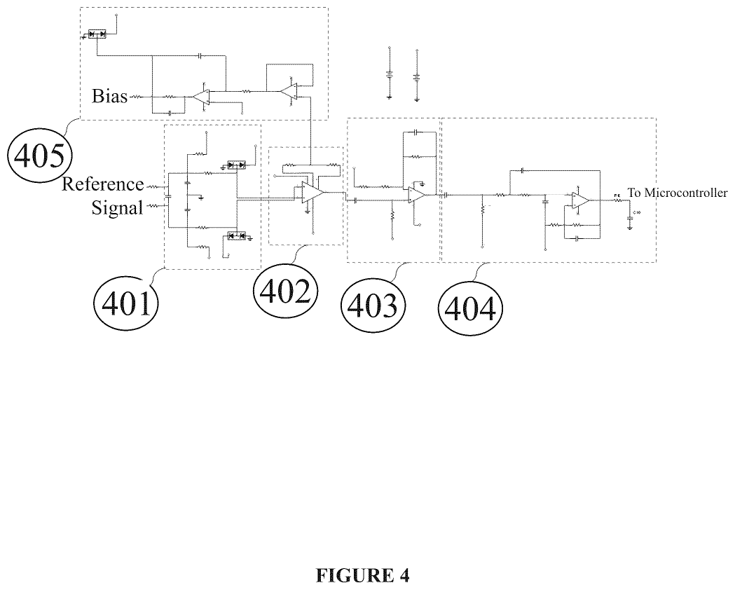

FIG. 4 exhibits the signal conditioning circuit.

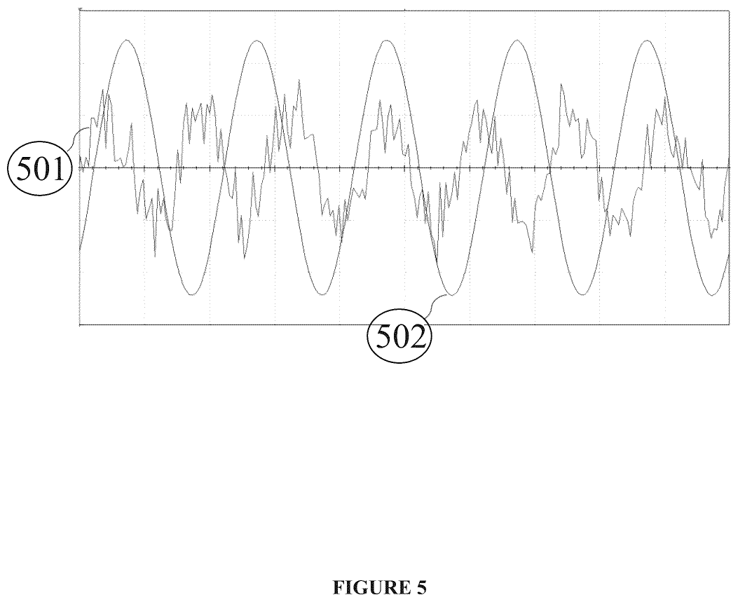

FIG. 5 displays the output of the Instrumentation Amplifier with an input of the raw EEG signal.

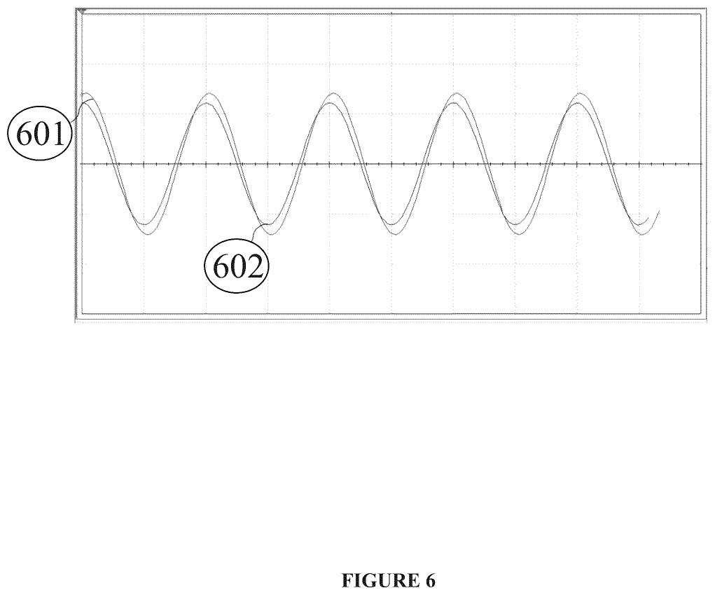

FIG. 6 displays the output of the active high pass filter.

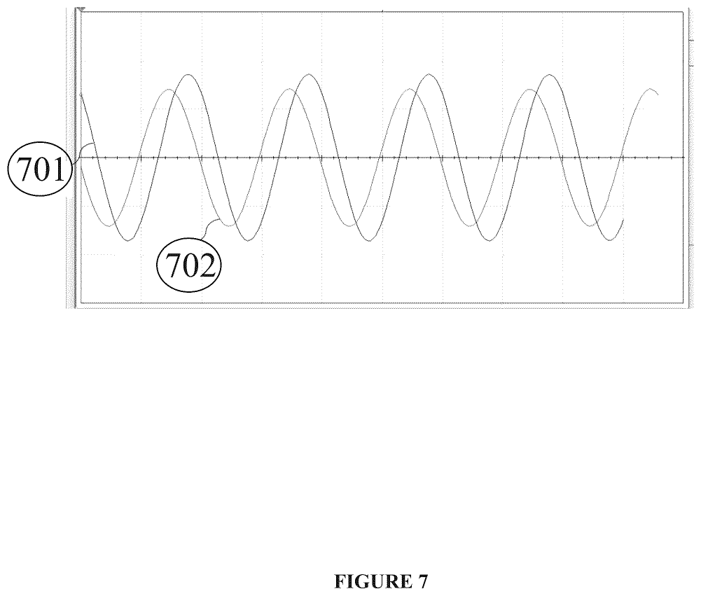

FIG. 7 displays the output of the signal condition block. The signal from here is transmitted to the microcontroller for further processing and analysis.

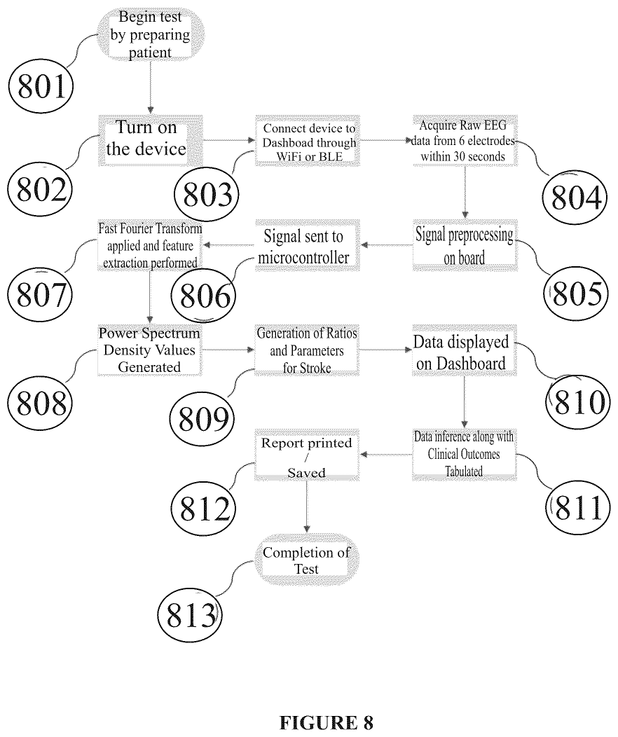

FIG. 8 shows a step-by-step flow chart of the algorithm to display the conclusive parameters.

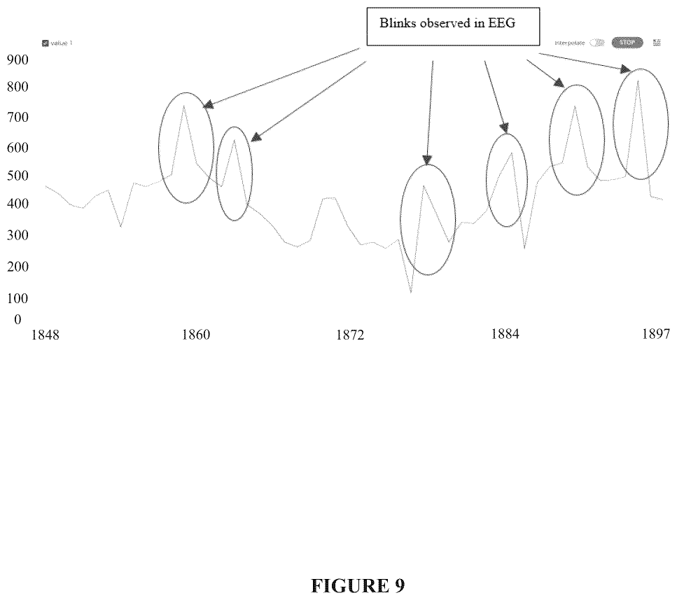

FIG. 9 shows the rachet mechanism deployed in the headset to accommodate the movable electrodes.

In at least an embodiment, the current invention is an EEG-based system that captures bioelectric signals from six electrodes placed according to the international 10:20 system of placement of electrodes to capture data. The signals are then analysed, by a signal processing engine, to derive frequency dependent ratios. The results of these computed parameters provide information for the following medical applications:
a) Stroke versus non-stroke mimic conditions during acute clinical assessment;
b) Ischaemic versus haemorrhagic stroke during acute clinical assessment;
c) Predict clinical recovery following confirmed stroke during acute clinical assessment;
d) Long Term Monitoring of patients having been diagnosed with Stroke, and/or administered with tPA.

This is a first of a kind, non-invasive, point-of-care, portable headset device, can distinguish strokes from stroke mimics with an estimated sensitivity of greater than 85% and specificity of greater than 85%. The sensitivity and specificity have been estimated based on an experimental setup that incorporated one EEG electrode on the prefrontal cortex with a 3-minute experimental setup and observed sensitivity and specificity of the device to be between the range of 85% and 90% for various qEEG parameters. The reference for the experiment was obtained against CT/MRI (Jeffrey M Rogers, 2019; Acute EEG Patterns Associated With Transient Ischemic Attack. Clin EEG Neurosci). Since the present invention incorporates higher number of electrodes to provide reliable signals with high fidelity, the sensitivity and specificity is estimated to be higher.

The present invention is able to distinguish between ischemic stroke and hemorrhagic stroke with an estimated sensitivity of 90% since qEEG based systems have shown to have an overall sensitivity of 91.7% for predicting any stroke, sensitivity of 90.3% for ischemic stroke, and sensitivity of 94.1% for hemorrhagic stroke (Matan Gottlibe, 2020; Stroke identification using a portable EEG device-A pilot study. Neurophysiologie Clinique) (Shreve L, K. A. 2019; Electroencephalography measures are useful for identifying large acute ischemic stroke in the emergency department. J Stroke Cerebrovasc Dis.) (Michelson E A, H. D. 2015; Identification of acute stroke using quantified brain electrical activity. Acad Emerg Med.).

The present invention aims at bridging the gap between low-end wireless EEG systems and high-end EEG systems by providing a solution which incorporates high accuracy in terms of ADC resolution, point of care “standalone” system. Since the data display is wireless, the setup and test time is less than 5 minutes. The present invention provides local and remote display of data which increases the user-friendliness and reduces the dependency of Wi-Fi networks for evaluation for the subject.

In at least an embodiment, of an EEG based device, of this invention, in order to capture stroke parameters, its system level architecture consists of three primary components:
an EEG based headset 101,
a dashboard 103, and
a communication protocol 102, to transfer data from the headset 101 to the dashboard 103.

Typically, the headset 101 consists of eight electrodes. Of these eight electrodes, 6 are signal electrodes, one reference electrode, and one bias electrode. The reference and the bias electrodes are positioned at a fixed location as a mastoid, behind a left ear and a right ear, respectively. The six signal electrodes are further bifurcated into four permanent position electrodes and two variable position electrodes. The placement of the electrodes follows the international 10:20 system of EEG electrode placement. The four permanent electrodes are positioned at O1, O2, T3, and T4. The movable electrodes are variable length electrodes. The mechanical design of the headset is constructed in a way that the movable electrodes can be positioned anywhere on the scalp to capture specific signals according to need. The extension and recoiling of the electrodes are achieved. This mechanism aids in easy release, strong affixation of the electrode in the desired position and seamless retraction of the electrode in the rachet mechanism.

In at least an embodiment, the headset 101 with 8 electrodes (consisting of 6 signal electrodes, 1 reference electrode, and 1 ground electrode) is used by a user. The 6 signal electrodes are, further, categorized into 4 fixed electrodes (located at O1, O2, T3, and T4 according to 10-20 international EEG system) and 2 variable electrodes, which can be placed at locations of interest. Raw EEG data is fed to the headset 101 and the data is collected for a fixed duration (minimum 30 seconds) based on use case/s. The raw data, initially, undergoes pre-processing before it is fed to the microcontroller.

Each electrode has at least two segments that contribute to raw signal acquisition, the two segments being:
a permanent part, and
a disposable electrode.

The disposable (preferably, Ag/AgCl) electrode is snapped in a permanent socket which is made of highly conductive material to ensure lossless transmission of the EEG signal. The disposable Ag/AgCl electrode serves two critical functions. Firstly, Ag/AgCl electrodes generate low noise level during biological signals recording (Mcadams, 2006). Secondly, the electrode consists of an adhesive that provides a strong support to the mechanical design; especially, the movable electrode. The signals acquired using the Ag/AgCl electrodes is strong, especially for EEG signals which are typically in uV and highly susceptible to noise.

The signals acquired by the headset 101 are sent, wirelessly 102, to the dashboard 103. The headset 101 provides preprocessing of signals, amplifying it to up to 10,000 times so as to maintain fidelity of signals. The 30 second data is collected in the headset 101 and is divided into epochs on which Fast Fourier Transform is performed. Feature extraction is performed on the frequency component of the signal and the stroke ratios are calculated on the headset 101, which is displayed on the dashboard 103. The dashboard 103 also has the feature of displaying the raw EEG signal in real time.

In at least an embodiment, of pre-processing stage, the pre-processing can be broken down into three stages. Since the EEG wave is very low in amplitude, generally, of 5 μV to 200 μV, it is difficult to handle the signal, protecting it from any type of noise interference.

In at least a first stage of pre-processing, there is a high precision amplifier which, typically, comprises an instrumentation amplifier. Its current-feedback input circuitry offers noise immunity along with high gain broad bandwidth. A gain of amplifier is set by settling the external resistor values between the gain ranges from 1 to 10,000.

In at least a second stage of pre-processing and third stage of pre-processing, there are provided are amplification stages that implement a third order butterworth bandpass filter. This filter removes unwanted high frequency noise since the filter is designed to operate between 0.16 Hz and 99.98 Hz.

The data received at a microcontroller is, thus, free of stray noise and is filtered and amplified.

A circuit level block diagram is depicted in FIG. 2. The block diagram consists of 5 distinct sections:
Power supply 201, 202, 203
Signal conditioning 204,
Microcontroller 206,
Display 207, and
Storage 208.

In at least an embodiment, the Power supply section consists of three independent blocks:
battery—The input source 201,
battery charging circuit 202, and
boost circuit 203.

In preferred embodiments, the battery 201 is a lithium-ion battery providing a stable DC power supply of 3.7 VDC. The battery 201 includes short circuit protection. The 3.7 VDC is fed to the boost circuit 203 which amplifies the DC signal to a stable 5 VDC. The boost circuit 203 is designed in a way that a constant source of 5 VDC would be provided to the circuit as long as the input to the boost circuit 203 is up to 1.8 VDC. Beyond 1.8 VDC, the boost circuit 203 shuts down. The battery is charged by an external power source. The battery charging circuit 202 consists of TP4056, a standalone constant-current/constant-voltage linear charger for single cell lithium-ion batteries. The power provided as an output from the boost circuit 203 is used to provide power to the signal conditioning circuit 204, microcontroller 206, display 207, and storage 208.

In at least an embodiment, the signal conditioning circuit 204 consists of six identical signal condition blocks 205. Each block consists of six input-output junctions:
Vcc (Power Supply) from the boost circuit 203,
Ground,
three inputs—Signal from the electrode, reference signal and bias signal, and
an output which is fed to the microcontroller 206.

The individual signal conditioning blocks 205 have very high Common mode rejection ratios (CMRR). The EEG signal obtained is, typically, in the range of microvolts. Being a very low magnitude signal, it is highly susceptible to external noise, motion artefacts and other high frequency noise. Thus, the primary focus of the signal conditioning circuit 205 is to amplify the EEG signal and to filter the noise signals; thus, maintaining the fidelity of the EEG signal for further analysis. Each signal conditioning block in the present invention provides an overall gain of 1500 and filters noise using multiple high pass, low pass, bandpass and notch filters.

The filtered signals from the signal conditioning block 204 are now in millivolts and are cleared of unwanted noise. These six signals are then sent to six independent analog-to-digital (ADC) channels of the microcontroller 206. The microcontroller 206 has 16-bit ADC channels for high precision digital conversion. The microcontroller 206 performs three critical functions:

Firstly, it receives the signal from the signal conditioning block 204, digitally filters the signals, performs data analysis techniques, and computes ratios, makes a probability analysis using comparative reference indices for stroke mimics, for the end use and stores the raw data along with the computed data on an external storage device 208.

Secondly, it displays the results locally on a display 207, which is an LED screen.

Thirdly, it, wirelessly, transmits the filtered data along with the computed ratios/indices to a dashboard through a cloud server or a local server.

The data received at the microcontroller 206 is free of stray noise and is filtered and amplified because of the three stages of pre-processing as explained above.

The key to precise analysis of the EEG waveforms is signal amplification and filtering. This function is carried out by the signal conditioning block 205. FIG. 3 elucidates the block diagram of the signal amplification and filtering system. The electrode deployed to capture the raw EEG signal consists of three electrodes: Signal, reference, and bias or ground. The ground is used for common mode rejection. The primary purpose of the ground is to prevent power line noise from interfering with the small biopotential signals of interest. By design, amplifiers should not be affected by large changes in potential at both the active and reference sites.

A ground electrode for EEG recordings is often placed on the forehead (but could be placed anywhere else on the body; the location of the ground on the subject is generally irrelevant). In the present invention, the ground electrode is placed on the mastoid behind the left ear.

Typically, the reference lead is the lead that connects the reference electrode; in EEG recordings, this electrode is usually placed at the ear or, in the case of “summed ears,” to a pair of electrodes, one at each ear. The measured electrical potential differences are ideally the voltage drops from the active electrode (connected to Vin+ on the amplifier) to the reference electrode (connected to Vin− on the amplifier). A reference electrode, is used to minimize power-line interference by means of decreasing the common-mode voltage obtained from the patient's body. In the present invention, the reference is placed at the mastoid behind the right ear. The “Signal” and the reference are given as input to the Vin+ and Vin− pins of the instrumentation amplifier 301. The instrumentation amplifier 301 has a very high CMRR and performs two functions:
amplification of the input signal, and
attenuation of high frequency noise.

After the signal has been filtered of unwanted noise, artefacts, white noise, line noise, and amplified by the instrumentation amplifier 301, the signal is fed to an active high pass filter 302. The function of the active high pass filter 302 is to allow the higher frequency signals to pass and to block the lower frequencies. In the present invention, the region of interest is frequencies in the range from 0.1 Hz to 100 Hz. The cut-off frequency fc of the high pass filter has been designed at 0.1591 Hz. The Active High pass filter circuit 302 also contributes to the amplification of the signal from input signal in the range of 100 uV to output signal in the range of 1 mV. The signal from the high pass filter 302 is then passed to a combination of bandpass filter, notch filter and non-inverting amplifier. The bandpass filter is realized with the help of a passive high pass filter 303 having a cut-off frequency again of 0.1591 Hz and a Low pass filter 305 having a cut-off frequency of fc=96.45 Hz. The notch filter and the non-inverting amplifier 304 is sandwiched in between the high pass filter 303 and the low pass filter 305. The notch filter removes the electrical line noise of 50 Hz and the non-inverting amplifier amplifies the signal with a gain of 18.434. Overall, the bandpass, notch and non-inverting amplifier provides a gain of 12.10.

The system level gain is observed to be 1504. This means that an input signal with Vp-p at 10 uV at the input of the instrumentation amplifier 301 gets amplified to 15.04 mV which is fed to the microcontroller 206.

The shield driving circuit 306 is interfaced with the ground electrode. A shielded drive circuit 306 is, typically, used to eliminate extraneous interferences on biopotential signal recordings, while also preserving all useful components of the target signal, in the present invention, the uV signals in the range of 0.1 Hz to 100 Hz.

FIG. 4 elucidates the actual circuit used to realize the block diagram explained in FIG. 3. The “signal” and “reference” biopotentials received from the electrodes is initially passed through protection circuit 401. This circuit ensures protection again electromagnetic interference and provides electromagnetic compatibility. The first amplification of the EEG signal is performed by the instrumentation amplifier circuit 402, which uses INA114 instrumentation amplifier, which offers excellent accuracy. The amplifier has a high common mode rejection ratio (CMRR) and is able to amplify the small signal difference down to microvolts range. The inputs of the instrumentation amplifier are the “signal” and the“Reference”. The signal is fed to Vin+ while the reference is fed to Vin−. The instrumentation amplifier amplifies the differential voltage of the two input signals, thereby removing noise from the circuit. The gain of the instrumentation amplifier circuit 402 can be calculated using the following equation:

$${A}_{INA} = 1 + 50 k / {R}_{G}$$

$$Where ,$$

$${A}_{INA} = Gain of the instrumentation amplifier$$

$${R}_{G} = External Gain setting resistor$$

In the present invention, RG has been designed to be 4.4 kohms. The effective instrumentation amplifier 301 gain is 12.36. Since the EEG signal can be both positive and negative, the present invention uses a virtual GND at the potential of 2.048V. This also helps reducing the noise from the common GND, improving the quality of the readings.

The output of the instrumentation amplifier circuit 402 is fed to the active high pass amplifier circuit. In order understand the DC and the AC component of the circuit, we perform DC analysis and AC analysis of the high pass filter circuit 402.

For DC analysis, all AC sources are short-circuited and all capacitors are open-circuited. The equation for computing the DC component of the High Pass Filter 403 is given as:

$${V}_{HPDC} = - {V}_{1} * ( \frac{R 12}{( R 9 + R 10 )} ) + {V}_{2} * ( \frac{R 12}{R 11} ) ( \frac{R 9 + R 10 + R 12}{( R 9 + R 10 )} )$$

$$Where ,$$

$$VHPDC = DC voltage of the high pass filter$$

$${V}_{1} = {V}_{2} = Virtual ground of 2.08 V$$

$$R 12 = 100 k ohm$$

$$R 10 = 1 k ohm$$

$$R 9 is a variable resistor$$

$$R 11 = 1 M ohm$$

Upon simulation, the DC voltage component of the high pass filter comes to 2.08V with the DC gain as 1.

For AC analysis, all DC sources are shorted to ground. The impedance of the capacitor is given by:

$$Xc = \frac{1}{2 π fC}$$

$$Where ,$$

$$Xc is the impedance contributed by the capacitor$$

$$f is the frequency of operation o the circuit$$

$$C is the capacitance$$

$$Xc for the High Pass filter circuit 403 is calculated as 3183.0985 ohms$$

The overall AC gain of the circuit can be computed as:

$${A}_{HPAC} = [ 1 + \frac{R 12}{R 9 + R 10} ] * [ \frac{R 11}{R 11 + Xc 4} ]$$

$$Where ,$$

$$AHPAC = AC gain for High pass filter$$

$$R 9 , R 10 , R 12 are gain setting resistors$$

$$R 11 is the High pass filter resistor$$

$$C 4 is the High pass filter capacitor$$

The overall AC gain of the high pass filter 403 is calculated as 10.058, with a cut-off frequency of 0.1591 Hz.

In at least an embodiment, the output of the High pass active filter 403 is sent first to another high pass filter with a cut-off frequency of 0.1591 Hz, followed by a notch filter of 50 Hz and a non-inverting amplifier, followed by a low pass filter with a cut-off frequency of 96.45 Hz. The High pass filter, the notch filter, the non-inverting amplifier and the low pass filter form blocks of the bandpass and notch filter block 404.

Upon performing DC and AC analysis in a method similar to the one explained for the High Pass Active Filter circuit 403, DC gain remains at unity. The AC gain of the high pass filter is 0.9968, the AC gain of the notch filter and non-inverting amplifier is 18.434 and the AC gain of the low pass filter is 0.6585. The overall AC gain for the bandpass and notch filter block 404 is a product of the individual AC gains and can be calculated as 12.10.

In at least an embodiment, a part of the signal from the instrumentation amplifier circuit 402 is fed to the shield driver circuit 405. The shield driving circuit 405 is interfaced with the ground electrode. A shielded drive circuit 405 is typically used to eliminate extraneous interferences on biopotential signal recordings, while also preserving all useful components of the target signal, in the present invention, the uV signals in the range of 0.1 Hz to 100 Hz.

FIG. 5 displays the input 501 and output 502 signals of the instrumentation amplifier 402. The input signal 501 is observed to be loaded with artefacts, noise, and other unwanted components. The output 502 of the instrumentation amplifier circuit 402 is observed as a clean signal with noise removed and having no harmonics. The signal also undergoes an amplification as well as a DC offset provided by the virtual ground of 2.08 VDC. The observed input signal 501 is in the range of 10 uV and the observed output signal 502 is in the range of 100 uV which validates the calculated gain of 12.36.

FIG. 6 displays the input 601 and output 602 of the high pass filter circuit 403. The input signal 601 is amplified and filtered. The signal is filtered with a cut off frequency of 0.1591 Hz and allows all signals greater than the cut-off frequency to pass into the circuit. The observed input signal 601 is in the range of 100 uV and the observed output signal 602 is in the range of 1 mV which validates the calculated gain of 10.058.

FIG. 7 displays the input 701 and output 702 of the bandpass and notch filter circuit 404. The input signal 701 is fed to a high pass filter with a cut off frequency of 0.1591 Hz and allows all signals greater than the cut-off frequency to pass into the circuit. The Gain of the high pass filter is 0.9968. The output of the high pass filter is sent to the notch filter of frequency 50 Hz to attenuate all the line noise. The notch filter can be adjusted to 60 Hz depending on the country in which the device is used. The signal is also amplified with an overall gain of the bandpass section as 18.434. The output of the notch filter is passed through the low pass filter, with a cutoff frequency of 96.45 Hz. The gain of the low pass filter is 0.6585. Thus, the signal is limited between 0.1591 Hz and 96.45 Hz with an overall gain of the bandpass and notch filter circuit as 12.10.

The EEG signal received is, further processed, in order to extract features using Quantitative EEG (QEEG) analysis. Quantitative electroencephalography (QEEG) is a modern type of electroencephalography (EEG) analysis that involves recording digital EEG signals which are processed, transformed, and analyzed using complex mathematical algorithms. QEEG has brought new techniques of EEG signals feature extraction:
analysis of specific frequency band and signal complexity,
analysis of connectivity, and
network analysis.

Clinical application of QEEG is extensive, including neuropsychiatric disorders, epilepsy, stroke, dementia, traumatic brain injury, mental health disorders, and many others.

The process of extracting features using QEEG analysis, from raw EEG data, requires systems with great computation power. In the present invention, the inventors have designed a system to perform this extensive computation on the headset itself, using techniques that can reduce computation requirement, considerably; thereby, ensuring that the screening system is a portable system and fidelity of the signal along with accuracy of results is not compromised on. This contributes to TECHNICAL ADVANCEMENT.

The time-based signals are initially digitized using a 15-bit analog to digital converter (ADC). This digital signal ranges from 0 to 32768, for 0V to 5V respectively. A raw EEG signal is provided with a DC offset to protect the EEG signal from noise. This DC offset is, then, removed after being digitized. Finite-duration Impulse Response (FIR) filter/s are one of the more popular type of filters implementation. For finite-duration impulse response (FIR) digital filter, its operation is governed by linear constant-coefficient difference equations of a non-recursive nature. A Finite-duration Impulse Response (FIR) filter is a filter structure that can be used to implement almost any sort of frequency response digitally. Although there are various window functions such as Blackman window function, Hanning window function, and rectangular window functions that can be used as digital filters, the Hamming window function was used, in a preferred embodiment, of this invention for a reason associated with its minimum damping/decibel of stopband with a reduced transition bandwidth.

FIG. 8 illustrates the system level flow chart of the invention.

A patient/subject is initially prepared for the test 801, by making the subject wear the headset and then turning the device “ON” 802. The device is then connected to the dashboard by a local WiFi network 803. On the device level, the 6 signal electrodes are connected to the designated locations. A total of 8 electrodes sense the EEG signals are fed to the headset. These 8 electrodes consist of six signal electrodes, one reference and one ground electrode. The six signal electrodes are again categorized into four fixed (located at O1, O2, T3, and T4 according to the 10-20 international EEG system) and two variable electrodes, which can be placed at locations of interest. The raw EEG data is fed to the headset and the data is collected for a fixed duration (minimum 30 seconds) 804 based on the use case. The raw data initially undergoes preprocessing 805 before it is fed to the microcontroller 806. The data received at the microcontroller 806 is free of stray noise and is filtered and amplified. Fast Fourier Transform (FFT) is performed on the data from each channel and feature extraction is performed 807. Using the values calculated from the Power Density Spectrum (Psd) 808 and executing brain rhythm quantification, a novel algorithm computes the Brain Symmetry Index (BSI) and frequency-specific power measures such as delta/alpha power ratio (DAR) and (delta+theta)/(alpha+beta) power ratio (DTABR) and other parameters 809. The algorithm to differentiate stroke from stroke mimics and between hemorrhagic and Ischemic strokes is embedded in the firmware. The qEEG parameters namely absolute and relative power of Delta (1 Hz-4 Hz), Theta (4 Hz-8 Hz), Alpha (8 Hz-12 Hz), Beta (12 Hz-30 Hz), DAR (Delta−Alpha Ratio), DTABR ((Delta+Theta)/(Alpha+Beta) Ratio), pdBSI (pairwise derived brain symmetry index) (Gratianne Rabiller J.-W. H., 2015; Perturbation of Brain Oscillations after Ischemic Stroke: A Potential Biomarker for Post-Stroke Function and Therapy. International Journal of Molecular Science) are calculated based on FFT and PSD. The unique algorithm assigns weightage to each of the parameters to compute a stroke index, and a Stype index (Stroke Type Index). On the basis of these indices, the microcontroller, provides the probability of the occurrence of stroke and the probability of the stroke being Ischemic in nature. If the subject/patient is suffering a stroke, only then will the algorithm compute the Stype index. This probability value gives the clinician the ability to plan the line of diagnosis and treatment. The real time data of EEG is displayed on the dashboard and the FFT data is computed. The brain rhythm parameters and analysis of the same to provide the Stroke index and Stype index are also computed on the microcontroller and displayed wirelessly on the dashboard created on a web server 810. The parameter inference range is displayed on the report and Clinical outcomes can be manually entered by the clinician 811. The report specifically mentions the probability of the disorder being stroke and the probability of the type of stroke being ischemic in nature. The generated report can then be saved or printed in multiple formats 812. With this final step, the testing procedure is complete 813. The clinician, patient and care-givers are provided with an end-to-end solution of stroke evaluation.

In at least an embodiment, Fast Fourier Transform (FFT) is performed on the filtered data from each channel. During the course of an FFT computation it is well known, according to prior arts, that to avoid loss of dynamic range, numerical issues must be dealt with at each butterfly computation stage, leading to a variety of trade-offs. For example, to minimize hardware resources, the word length could be kept same by rounding results after appropriate groups of operations, but in this case dynamic range would suffer. Alternatively, word lengths could grow a fixed number of bits after each butterfly operation to accommodate worst case numerical properties; however, for normal inputs, the extra hardware often is not required. Another common approach, according to prior arts, although it is usually adds more circuit hardware, is to use a “block” floating-point number representation. In this case the intermediate results are scaled together only if there is overflow is detected in some part of the circuit. At the end of the computation the total scaling is represented by a single exponent that is associated with each output FFT block of data. This is often a good compromise between the first two strategies.

In at least an embodiment, of the current invention, extensive computation on a portable hardware is performed by fast implementation of a standard FFT algorithm which operates on only real data. It can output up to 256 frequency bins at 16b depth, at a minimum of ˜7 ms update rate. The speed improvements in this particular implementation are due to two things:

First (leading to a first set of time savings), in any FFT, one must multiply the input variables by fixed cosine and sine constants. This process consumes the most time on any microcontroller or microcomputer, as 16b×16b multiplies take around 18 clock cycles. On the other hand, 16b+16b adds only take 2 clock cycles. So, it is better to add than it is to multiply. As it turns out, a lot of those sine and cosine constants used in the FFT are 0 or 1, so there is no requirement of multiplying and the data can be simply added. The firmware checks for that 0 or 1 condition, and simply performs addition instead. as it turns out, those constants occur at regular intervals, and can be easily checked for. The benefits of this sort of approach are limited for larger FFTs. The total savings is (1.5*N−2) for an N sized FFT, whereas the total number of multiplies is (N/2)*log 2 (N). This gives a savings ratio of 3/log 2 (N), which drops as N increases.

Second (leading to a second set of time savings), in turn computation management, in this implementation comes from using lookup tables to calculate the square roots of the magnitudes. The difficulty in this method is that input mapping to a lookup table is much larger than actual contents of the lookup table itself. So as to not waste memory space, a compression of the input values is done.

The above also contributes to TECHNICAL ADVANCEMENT.

In at least an embodiment, once the FFT is computed, complex real and imaginary values computed are converted to magnitudes, which are further converted to decibels.

In the present invention, according to a preferred embodiment, number of samples is 1024 and sampling frequency is 256 Hz. As such, power value is recorded at an interval of 0.25 Hz.

In at least an embodiment, signals, from each of the 6 electrodes, are captured and power that is computed is broken into 5 baskets. Initially, for each electrode:
a delta basket, which adds all power values from 0 Hz to 4 Hz (power values obtained at intervals of 0.25 Hz),
a theta basket, which adds all power values from 4 Hz to 8 Hz,
an alpha basket which adds all power values from 8 Hz to 12 Hz,
a beta basket which adds all power values from 12 Hz to 30 Hz, and
a total power basket, which adds all power values from 0 Hz to 30 Hz.

Similar calculations are performed for the other 5 electrodes, namely FP2, T3, T4, O1 and O₂.

Further, relative power for each of spectral bands (Delta: 0-4 Hz; Theta: 4-8 Hz; Alpha: 8-12 Hz; Beta: 12-30 Hz) is calculated for each channel. Relative powers were obtained by normalizing with a total power across the 0-30 Hz range. In addition, (delta+theta)/(alpha+beta) ratio (DTABR) and delta/alpha ratio (DAR) were computed. Relative power for each band, DAR and DTABR parameters were averaged over all 6 scalp electrodes.

On the basis of multiple studies carried out by scientists in the field of DAR and DTABR as indices to reflect Stroke, it has been established that DAR has 100% sensitivity and 100% specificity to stroke. Other prior arts established DTABR with 96% sensitivity and 100% specificity to stroke.

The current invention is based primarily on these two parameters: DAR and DTABR.

The current invention's system and method combines DAR and DTABR to give Stroke Index.

$$Stroke Index ( SI ) = 0.75 * DAR + 0.25 * DTABR$$

A weighted average of DAR and DTABR are computed to give the Stroke Index.

A probability of Stroke is computed based on value of Stroke Index. A lookup table is generated that provides the probability of Stroke based on Stroke Index.

According to prior art, it has been established that a DAR value of greater than or equal to 3.7 is 100% sensitive and specific to stroke and a DTABR value greater than 1.76 is 96% sensitivity and 100% specificity to stroke.

Using these threshold values, the following lookup table is established:

| Stroke Index (SI) | Percentage Probability of Stroke |
|---|---|
| SI ≤ 2.85 | 0% |
| 2.85 < SI ≤ 2.90 | 25% |
| 2.90 < SI ≤ 2.95 | 35% |
| 2.95 < SI ≤ 3.00 | 45% |
| 3.00 < SI ≤ 3.05 | 55% |
| 3.05 < SI ≤ 3.10 | 65% |
| 3.10 < SI ≤ 3.15 | 75% |
| 3.15 < SI ≤ 3.20 | 85% |
| 3.20 < SI ≤ 3.215 | 95% |
| SI > 3.215 | 100% |

In order to differentiate between Ischemic Stroke and Haemorrhagic stroke, tt has been observed that In case of Haemorrhagic stroke, The percentage of Relative Delta Power is strongly negatively correlated to the cerebral Blood Flow (CBF) and Relative Alpha Power is Strongly positively correlated.

In Ischemic stroke, the values of RDP and RAP change, however, the difference from Normal is not as drastic as observed in Haemorrhagic stroke.

The stroke type Index, Stype, is a weighted average of RDP and RAP.

RDP is calculated as the percentage of delta power of the total power and RAP is calculated as the percentage of alpha power of the total power.

$$Stype = 0 \text{.7} * RDP + 0 \text{.3} * RAP$$

The probability of Hemorrhagic stroke can be observed from the following table:

| | Percentage probability of Haemorrhagic |
|---|---|
| Stroke Type Index (Stype) | Stroke |
| 45 ≤ Stype ≤ 48 | 40% |
| 48 < Stype ≤ 52 | 60% |
| 52 < Stype ≤ 54 | 80% |
| 54 < Stype ≤ 57 | 90% |
| 57 < Stype ≤ 58.5 | 95% |
| Stype > 58.5 | 100% |

Stype is computed only if the subject has been confirmed of stroke with the help of stroke index (SI)

FIG. 9 shows the rachet mechanism deployed in the headset to accommodate the movable electrodes.

Typically, the headset 101 consists of eight electrodes. Of these eight electrodes, 6 are signal electrodes, one reference electrode, and one bias electrode. The reference and the bias electrodes are positioned at a fixed location as a mastoid, behind a left ear and a right ear, respectively. The six signal electrodes are further bifurcated into four permanent position electrodes and two variable position electrodes. The placement of the electrodes follows the international 10:20 system of EEG electrode placement. The four permanent electrodes are positioned at O1, O2, T3, and T4. The movable electrodes are variable length electrodes. The mechanical design of the headset is constructed in a way that the movable electrodes can be positioned anywhere on the scalp to capture specific signals according to need. The extension and recoiling of the electrodes are achieved using a micro-rachet mechanism 901.

The micro-rachet is based on a retractable mechanism. The permanent part 902 is coiled around within the rachet 901. Upon extending the electrode 902 to place it at the desired location with the help of the disposable Ag—AgCl electrode 903, the spring in the rachet 901 is compressed and stores potential energy. Once the test is completed, the retractable electrode is released, which in turn releases the stored potential energy in the spring and retracts the electrode in its original coiled position in the rachet 901 of the housing. This mechanism aids in easy release, strong affixation of the electrode in the desired position and seamless retraction of the electrode in the rachet 901.

The process is differentiated by prior EEG-based processes because of at least the following reasons:
i. From a hardware perspective, the system, of this invention, involves a unique method for cleaning the signals of artefacts and noise, with the implementation of instrumentation amplifier with high common mode rejection ratio (CMRR). The system, of this invention, involves amplification of signal up to 10,000 times, from raw EEG signal obtained in the range of 10 uV to the input to the microcontroller in the range of 100 mV. Thus, the qEEG signals obtained at the microcontroller are therefore more precise and accurate than other point of care EEG devices available.
ii. Once the signals are received at the microcontroller, Fast Fourier transformation (FFT) is computed on the microcontroller. Since the signal analysis and parameter computation is performed at the microcontroller level, noise induced during transmission of raw EEG signal to a third system is avoided. The computed parameters along with the processed EEG signals are then sent to a third system for display only. This gives an added advantage over already existing EEG devices where computation takes place on another system.
iii. The computation of the quantitative EEG and other power density spectrum parameters are executed, in the firmware, of the current invention, at the microcontroller level. This empowers the EEG based headset to act as a stand-alone system having advantage in geographical locations where there are limitations of network. An LED display on the headset itself, allows the headset to independently capture, process, analyze and display EEG signals and its computed parameters
iv. The software logic is unique, whereby it displays on a customized dashboard the signals, the FFT results, ratios, and final stroke/non-stroke analysis along with a probability value of the stroke outcome.
v. The device is unique in the context of the outcome parameters it is providing-Stroke Index and Stype Index. The Stroke Index provides a probability of the patient suffering from stroke, differentiating it from a stroke mimic. The Stype Index provides a probability of the patient suffering from an Ischemic Stroke or a hemorrhagic stroke.

The present technology stands out in multiple novel features as compared to the prior art:
Computation of Fast Fourier Transform (FFT) on board is a unique feature of this current invention. The microcontroller collects the signal, every millisecond, and stores it in an array. This data is sampled at 256 Hz and FFT is performed on it on the microcontroller. The FFT values are plotted on a power spectrum density plot and the power for each of the qEEG parameters is computed, which is locally displayed on an LED screen and transmitted wirelessly over Wi-Fi to a web server. The above process is unique and allows the headset to work as an independent unit.
The algorithm to differentiate stroke from stroke mimics and between hemorrhagic and Ischemic strokes is unique to the present invention. The qEEG parameters namely absolute and relative power of Delta (1 Hz-4 Hz), Theta (4 Hz-8 Hz), Alpha (8 Hz-12 Hz), Beta (12 Hz-30 Hz), DAR (Delta-Alpha Ratio), DTABR ((Delta+Theta)/(Alpha+Beta) Ratio), pdBSI (pairwise derived brain symmetry index) (Gratianne Rabiller J.-W. H., 2015) are calculated based on FFT and PSD. The unique algorithm assigns weightage to each of the parameters to compute a stroke index, and a Stype index (Stroke Type Index). On the basis of these indices, the microcontroller, provides the probability of the occurrence of stroke and the probability of the stroke being Ischemic in nature. If the subject/patient is suffering a stroke, only then will the algorithm compute the Stype index. This probability value gives the clinician the ability to plan the line of diagnosis and treatment.
The headset is designed to act as a stand-alone system that captures the raw signals, filters and amplifies it (preprocessing), computes the brain rhythm parameters and displays the output results locally on a display on the headset. Thus, the adaptability of the device in rural locations, where availability of network/systems to display the data might not be feasible, is considered and a solution for the same is provided.
The device performs signal processing and filtering at two levels, the hardware as well as the firmware level. The hardware signal processing ensures the filtering and amplification of the raw signal, maintain the integrity of the signal. The microvolt input EEG signal needs 1000-10000 times amplification, which is performed by the hardware signal processing circuit. The firmware signal processing and filtering ensures removal of noise induced on the board level.
The novel design of incorporation of fixed and variable electrodes: Having a set of fixed and variable electrodes serves two functions. The fixed electrodes provide convenience for the clinician to ensure that the electrode placement is not a concern. Thus, the highly skilled task of ensuring the correct placement of electrodes is taken care of by the device, making it user-friendly, especially in a rural setup. The variable electrodes provide flexibility to the clinician to measure signals at different locations, depending on the case.
The design has been optimized to be used as a quick screening tool at an emergency room or ambulatory care and for long term use in an ICU set-up.
The device transfers data wirelessly and through a webserver. Since the data is transmitted over the internet, secured accessibility of the data for clinicians at remote locations is established. The dashboard generals a clinical report with the necessary fields including the actual signal, the qEEG PSD components and the computed parameters, along with necessary references and the reference range. The dashboard also displays the probable outcome based on a unique algorithm and gives provision for the clinician to comment and then print the entire report in multiple formats.
On-board storage of data is made available with the access to a microUSB port.
Combination of wet and dry electrodes. The electrodes are Ag—AgCl electrodes that are one-time useable electrodes. These electrodes have Ag—AgCl gel pre-filled that avoids the conventional EEG methodology of inducing the gel once the electrode has been placed. The adhesive in the electrodes of the present invention ensures that the electrode is stationary, reducing motion artefacts. The Ag—AgCl provides a capacitive-resistive chemical filter that removes noise at the time of signal acquisition.

The TECHNICAL ADVANCEMENT of this invention lies in providing a stand-alone non-invasive system for early detection of stroke and its subtypes using electroencephalography (EEG). The TECHNICAL ADVANCEMENT of this invention lies in providing systems and methods for early detection of stroke, differentiating it from stroke-mimics, and distinguishing between stroke subtypes namely Ischemic and Hemorrhagic stroke.

According to a non-limiting exemplary embodiment, the following experiments were conducted: Blink Test as a Valid Method for Verifying the Functionality of the EEG System deployed using the system and method of this invention.

Electroencephalography (EEG) is a widely used method for monitoring brain activity, making the verification of its functionality crucial in various research and clinical settings. The blink test, involving the measurement of eye blinks in response to specific stimuli, has gained attention for its potential utility in assessing EEG system performance. When evaluating the functionality of an EEG system, it is essential to consider the quality of the data captured under different conditions, including mobile or ecologically-valid environments. As highlighted in recent research (Bin et al.), the study assessing steady-state visual-evoked potentials (SSVEPs) using a mobile EEG system revealed potential challenges when subjects transitioned from stationary to walking states. This indicates the impact of natural human behaviours, such as locomotion, on EEG signal quality. Furthermore, referencing a dataset of neonatal EEG recordings (Boylan et al.), which graded abnormalities in background patterns, provides insight into the importance of assessing EEG attributes like amplitude, frequency, and continuity for evaluating system performance. By understanding the effects of varying conditions on EEG signals and considering grading systems for abnormalities, researchers can develop comprehensive methods for assessing EEG system performance, thus addressing the central question of using the blink test as a reliable measure for EEG functionality in diverse settings.

The blink test, thus can indeed be used as a method to verify the functionality of the EEG system deployed using the system and method of this invention.

### Objective:

The objective is to ensure accurate detection and analysis of blink-related EEG signals for research and clinical applications, to be used in detection of stroke using the system and method of this invention.

### Materials:

System and method of this invention (including electrodes, headset, and software)
Electrode Gel or Paste
Computer for Data Acquisition and Analysis
Comfortable Chair or Bed for Participant
Stopwatch or Timer
Eye Blink Cue (visual or auditory)

### Preparation:

Set up the invention's EEG system in a quiet, well-lit room with minimal electromagnetic interference.

Ensure all components of the system are properly connected.

Verify electrode impedance positions.

Prepare electrodes by applying conductive gel or paste to ensure good electrode-skin contact.

### Participant Preparation:

Position the participant comfortably in a chair or on a bed.

Clean the participant's forehead and areas where electrodes will be placed to remove any oils or dirt.

Apply electrodes according to the international 10-20 system, ensuring proper placement for blink detection (e.g., above and the eyes on FP1 and FP2 electrode positions according to the 10-20 system).

### Blink Test Procedure:

Start the invention's EEG recording session and ensure stable baseline signals.

Instruct the participant to keep their eyes open and relaxed for the initial baseline period (e.g., 1-2 minutes).

Introduce the blink task cue (visual or auditory) to prompt the participant to blink voluntarily.

Record EEG signals while the participant blinks naturally in response to the cue.

Repeat the blink task multiple times with sufficient breaks between trials to prevent fatigue.

### Data Analysis:

The blink test provides a four-point verification as stated below:
i. Signal Acquisition Verification: Conducting a blink test allows for the real-time observation of EEG signals associated with blinking. If the EEG system is functioning properly, it should accurately capture these blink-related electrical activities. Observing the expected EEG patterns in response to blinking confirms that the system is correctly acquiring and recording brain signals.
   In FIG. 9, the inventors could clearly observe the sharp spikes when the stimuli of a blink was provided.
ii. Artifact Detection: Blink artifacts are a common challenge in EEG recordings. Verifying the ability of the EEG system to detect and differentiate blink artifacts from genuine brain activity is essential.
iii. System Calibration: Before conducting experiments or clinical assessments using EEG, it's important to ensure that the system is calibrated and optimized for accurate signal acquisition. Performing a blink test serves as a calibration step by providing baseline data on blink-related EEG patterns.
iv. Quality Assurance: Incorporating the blink test into routine quality assurance procedures helps verify the reliability and consistency of the EEG system over time. Regularly testing the system's ability to detect blink artifacts ensures that it remains in proper working condition and maintains its accuracy for research and clinical applications.

Overall, the blink test served as a valuable tool for verifying the functionality, accuracy, and reliability of this invention, making it an essential component of system validation and quality assurance processes in EEG research and clinical practice, especially as a precursor to establishing a system for early detection of stroke.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A system for differentiating between stroke and stroke mimics with electroencephalography (EEG), the system comprising:
electrodes configured to detect and transmit EEG signals for time durations while placed on a patient, wherein each electrode includes a permanent part segment and a disposable electrode segment;
a client-side signal processing engine deriving frequency-dependent ratios from the EEG signals by being configured to,
receive the EEG signals from each of the electrodes,
amplify the EEG signals to obtain amplified signals,
divide the amplified signals into epochs,
perform a Fast Fourier Transform on the epochs to obtain transformed signals,
process, with a signal conditioning circuit including identical signal condition blocks having a high common-mode rejection ratio (CMRR), the transformed signals, wherein the processing outputs amplified signals and processed signals with attenuated high frequency noise parameters; and
a dashboard configured to receive data from the client-side signal processing engine at a headset containing the electrodes.

2. The system of claim 1, wherein the client-side signal processing engine is further configured to,
receive, with independent analog-to-digital (ADC) channels, corresponding signals from the signal conditioning circuit to compute power for the processed signals and segregate each processed signal, from each of the electrodes, into five baskets, wherein,
a Delta basket adds all power values from 0 Hz to 4 Hz with their power values obtained at intervals of 0.25 Hz, with a relative power spectral band being in the region of 0-4 Hz,
a Theta basket adds all power values from 4 Hz to 8 Hz with their power values obtained at intervals of 0.25 Hz, with a relative power spectral band being in the region of 4-8 Hz,
an Alpha basket adds all power values from 8 Hz to 12 Hz with their power values obtained at intervals of 0.25 Hz, with a relative power spectral band being in the region of 8-12 Hz,
a Beta basket adds all power values from 12 Hz to 30 Hz with their power values obtained at intervals of 0.25 Hz, with a relative power spectral band being in the region of 12-30 Hz,
extract features from a frequency component of the transformed signals to obtain stroke ratios,
receive, as a first output, a first set of processed signals with power ratings including, a first power rating ratio (DTABR) as a function of ratios of the delta basket and the theta basket to the alpha basket and the beta basket, a second power rating ratio (DAR) as a function of ratios of the delta basket and the alpha basket, and a determination of a stroke incident as a function of the first power rating ratio and the second power rating ratio,
receive, as a second output, a second set of processed signals including, a first relative power (RDP) as a function of powers of signals from the delta basket, a second relative power (RAP) as a function of powers of signals from the alpha basket, and a determination of a type of stroke as a function of the first relative power (RDP) and the second relative power (RAP).

3. The system of claim 1, wherein the electrodes include at least six signal electrodes, one reference electrode, and one bias electrode.

4. The system of claim 1, wherein, the electrodes include four fixed signal electrodes, two variable signal electrodes, one reference electrode, and one bias electrode.

5. The system of claim 1, wherein the disposable electrode segment is an Ag/AgCl electrode snapped in a permanent socket made of conductive material to ensure lossless transmission of the EEG signal.

6. The system of claim 1, wherein the amplification is in the region of 10,000 times to maintain fidelity of signals.

7. The system of claim 1, wherein the client-side signal processing engine further includes a pre-processing engine with a first-stage pre-processor having a high precision amplifier with a gain set by setting external resistor values between gain ranges from 1 to 10,000 and with a second-stage pre-processor, and a third-stage pre-processor including amplification stages, filters removing unwanted high frequency noise configured to operate between 0.16 Hz and 99.98 Hz, wherein the pre-processing engine is configured to filter signals with regions of interest of frequencies being in the range from 0.1 Hz to 100 Hz.

8. The system of claim 1, wherein the client-side signal processing engine includes a pre-processing engine configured with instructions to,
first-stage pre-process, with a high precision amplifier with a gain set by setting external resistor values between gain ranges from 1 to 10,000, wherein the amplifier includes an active high pass filter circuit contributing to the amplification of the signal from an input signal in the range of 100 μV to an output signal in the range of 1 mV, and
second-stage pre-process and third-stage pre-process, with amplification stages and filters for removing unwanted high frequency noise, wherein the filters are configured to operate between 0.16 Hz and 99.98 Hz and include a bandpass filter, a notch filter, and a non-inverting amplifier, wherein the bandpass filter being includes a passive high pass filter having a cut-off frequency of 0.1591 Hz, and a low pass filter having a cut-off frequency of 96.45 Hz, the notch filter and the non-inverting amplifier being positioned between the high pass filter and the low pass filter, wherein the notch filter is configured to remove electrical line noise of 50 Hz, the non-inverting amplifier is configured to amplify the signals with a gain of 18.434, and the bandpass filter, the notch filter, the and non-inverting amplifier together provide an overall gain of 12.10.

9. The system of claim 1, wherein the processed signals, from each of the six electrodes are classified into,
a delta spectral band, which adds all power values from 0 Hz to 4 Hz (power values obtained at intervals of 0.25 Hz),
a theta spectral band, which adds all power values from 4 Hz to 8 Hz,
an alpha spectral band which adds all power values from 8 Hz to 12 Hz,
a beta spectral band which adds all power values from 12 Hz to 30 Hz, and
a total power spectral band, which adds all power values from 0 Hz to 30 Hz.

10. The system of claim 9, wherein the server-side signal processing engine includes a processor to,
determine relative power for each spectral band for each channel, each channel having six electrodes,
obtain relative powers by normalizing with a total power across the 0-30 Hz range,
compute a first ratio being a DTABR ratio,
compute a second ratio being a DAR ratio,
average relative power for each spectral bank, each of the first ratios and each of the second ratios, and
compute a stroke index (SI) as a weighted function of the first ratio and the second ratio.

11. The system of claim 1, wherein the client-side signal processing engine includes a pre-processing engine configured to,
first-stage pre-processing, comprising a high precision amplifier with its gain set by setting external resistor values between gain ranges from 1 to 10,000, the amplifier comprising:
Active High pass filter circuit contributing to the amplification of the signal from input signal in the range of 100 uV to output signal in the range of 1 mV;
second-stage pre-processing, and third-stage pre-processing comprising amplification stages, filters removing unwanted high frequency noise, the filters being designed to operate between 0.16 Hz and 99.98 Hz, wherein the filters include a bandpass filter, a notch filter, and a non-inverting amplifier, output of the High pass active filter is sent first to another high pass filter with a cut-off frequency of 0.1591 Hz, followed by a notch filter of 50 Hz and a non-inverting amplifier, followed by a low pass filter with a cut-off frequency of 96.45 Hz.

12. The system of claim 1, wherein the client-side signal processing engine includes a pre-processing engine configured to,
first-stage pre-process, with a high precision amplifier with a gain set by setting external resistor values between gain ranges from 1 to 10,000, wherein the amplifier includes, a Active High pass filter circuit contributing to the amplification of the signal from input signal in the range of 100 uV to output signal in the range of 1 mV, and
second-stage pre-process, and third-stage pre-processing comprising amplification stages, filters removing unwanted high frequency noise, the filters being designed to operate between 0.16 Hz and 99.98 Hz, wherein the filters include a bandpass filter, notch filter and non-inverting amplifier, the AC gain of the high pass filter is 0.9968, the AC gain of the notch filter and non-inverting amplifier is 18.434 and the AC gain of the low pass filter is 0.6585.
